(19) United States
(12) Patent Application Publication
(10) Pub. No.: US 2007/0276152 A1
(43) Pub. Date: Nov. 29, 2007

(54) Aromachemicals

(75) Inventor: Luca Turin, London (GB)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Flexitral, Inc., Chantilly, VA (US)

(21) Appl. No.: 11/695,472

(22) Filed: Apr. 2, 2007

Related U.S. Application Data
(60) Provisional application No. 60/788,063, filed on Apr. 3, 2006.
(60) Provisional application No. 60/788,064, filed on Apr. 3, 2006.
(60) Provisional application No. 60/827,913, filed on Oct. 3, 2006.

(51) Int. Cl. A23L 1/22; A61K 8/40; A61Q 13/00; C07C 255/01; C11D 3/26

(52) U.S. Cl. 558/303; 426/534; 510/108; 510/276; 512/6

(57) ABSTRACT

A compound or mixture of compounds of formulae: 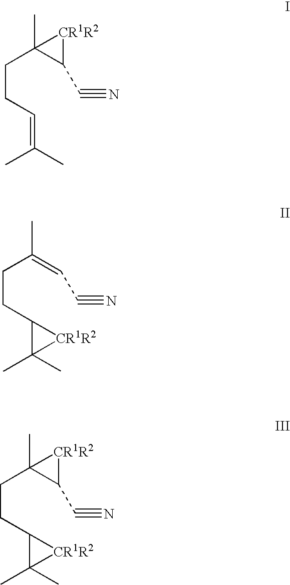
wherein R¹ and R² are each independently H or CH₃.

### CROSS REFERENCE TO RELATED APPLICATIONS

The present application claims priority to Provisional Application Nos. 60/788,063, filed Apr. 3, 2006, 60/788,064, filed Apr. 3, 2006 and 60/827,913 filed Oct. 3, 2006, the contents of each are hereby incorporated by reference in their entireties.

### FIELD OF THE INVENTION

The present invention relates generally to the field of flavors and fragrances. More particularly, the present invention relates to derivatives of conventional compounds that provide perfumes and other articles with properties and advantages not shared by the conventional compounds from which they are derived. These derivatives find utility in any and all applications requiring flavours and fragrances. The invention also provides mixtures of these derivatives, methods for their preparation and their use as perfume materials for application in a variety of substrates and their use in flavoring and articles of manufacture and compositions including the derivatives.

### BACKGROUND OF THE INVENTION

There are a large number and variety of known flavors and fragrances used as ingredients in perfumes and in a varied range of other products. However, many aromachemicals include double bonds and/or other reactive groups that are potentially susceptible to reaction and may result in a limited useful lifetime. Further, many essential oil fragrances have recently been determined to have undesirable properties which mean they are potentially harmful to human health. For example, some cause allergic reactions, while some may be toxic or genotoxic, for example carninogenic. It is becoming increasingly difficult to bring products containing such fragrances to market. There is also a demand for new flavours and fragrances that have novel or improved fragrance profiles and/or other properties that make them particularly useful for use as fragrances and/or flavours.

The listing or discussion of a prior-published document in this specification should not necessarily be taken as an acknowledgement that the document is part of the state of the art or common general knowledge.

### SUMMARY OF THE INVENTION

This invention provides new flavour and fragrance compounds that have novel and/or improved fragrance profiles. The flavours and fragrances compounds also have one or more additional properties that make them particularly suitable for use as fragrances and/or flavours. Also provided are methods by which to make the novel flavours and fragrances compounds.

According to one aspect of the invention, there is provided one or more compounds of formulae: 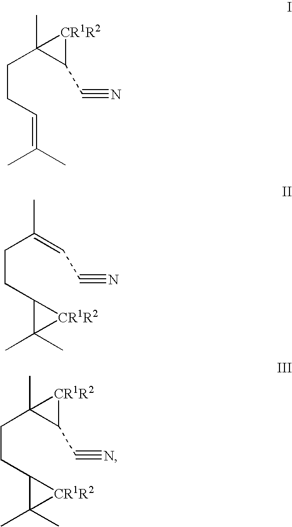
wherein R¹ and R² are each independently H or CH₃.

In one embodiment, there is provided one or more compounds having the following formulae:

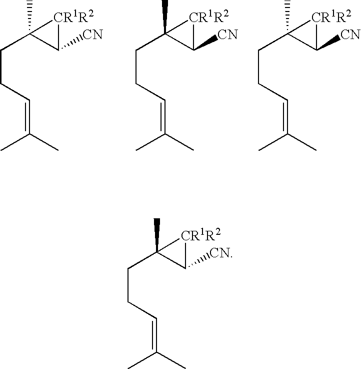

In yet another embodiment, there is provided one or more compounds having the following formulae:

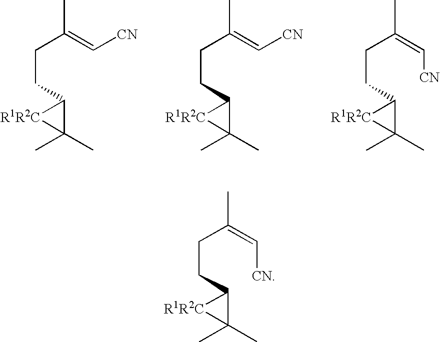

In still another embodiment, there is provided one or more compounds having the following formulae:

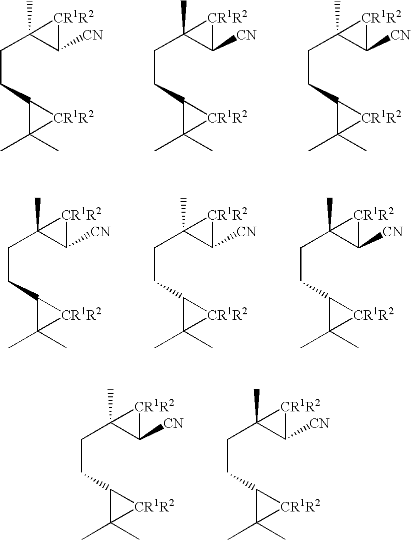

According to another aspect of the invention, there is provided a method for preparing a compound or mixture of compounds comprising converting at least one aldehyde compound of formulae

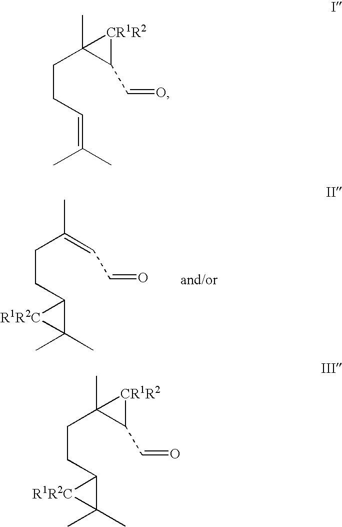

to a compound or mixture of compounds of formulae:

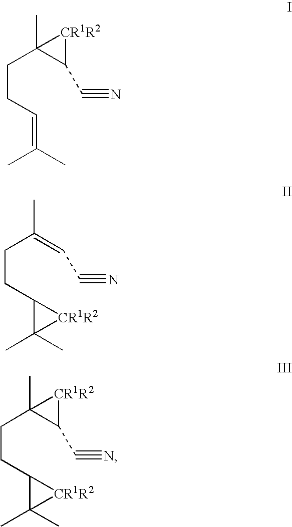

wherein R¹ and R² are each independently H or CH₃.

In a preferred embodiment, the method includes cyclopropanating geranial and/or neral to produce the compound or mixture of compounds of formulae I″, II″ and/or III″.

In another preferred embodiment, the method includes oxidizing a compound or mixture of compounds of formulae

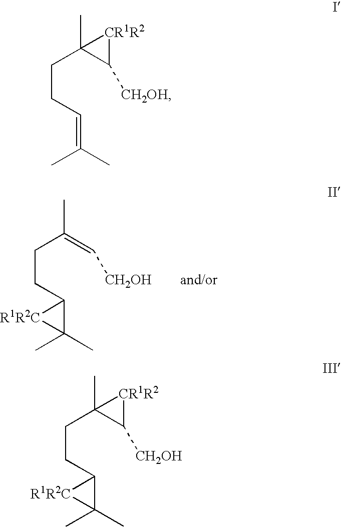

to produce at least one compound of formulae I″, II″ or III″.

In still another embodiment, the method includes cyclopropanating geraniol and/or nerol to produce the compound or mixture of compounds of formulae I′ or II′ or III′.

In another embodiment, the method includes cyclopropanating the compound or mixture of compounds of formulae I or II to produce the compound of formula III.

In yet another embodiment, there is provided a method for preparing a compound of the formula I, II or III, comprising cyclopropanating 3,7-dimethyl-2,6-octadienenitrile.

There is also provided a composition containing one or more of the compounds of the formula I, II or III, with the composition being in the form of a flavour and/or fragrance composition; a treated substrate; a composition having aroma, fragrance or odour releasing characteristics; a perfume, fragrance or cologne; a soap; a bath or shower gel; a hair care product; a cosmetic preparation; a body odorant, deodorant or antiperspirant; an air freshener; a liquid or solid fabric detergent or softener; bleach product; disinfectant or an all-purpose household or industrial cleaner.

There is also provided a composition containing one or more of the compounds of the formula I, II or III, with the composition being in the form of a beverage; a flavouring; a food; a chewing gum; a pharmaceutical; an orally-deliverable matrix material.

In another embodiment, there is provided a method to confer, improve, enhance or modify a taste or flavour property of a composition which comprises adding thereto a flavour effective amount of the formula I, II or III.

In yet another embodiment, there is provided a method to confer, improve, enhance or modify an aroma, fragrance or odour characteristics of a composition which comprises adding thereto an aroma, fragrance or odour effective amount of a compound the formula I, II or III.

In another embodiment, there is provided an article of manufacture comprising packaging material and an aroma, odour, fragrance, taste or flavour enhancing agent contained within the packaging material, wherein the agent is effective for the enhancement of the aroma, odour, fragrance, taste or flavour of a composition to which it is added, and wherein the packaging material comprises a label which indicates that the agent can be used for enhancing aroma, odour, fragrance, taste or flavour, and wherein the agent is a compound or mixture of compounds of the formula I, II or III.

### DETAILED DESCRIPTION OF THE INVENTION

The present invention provides a compound or mixture of compounds of formulae:

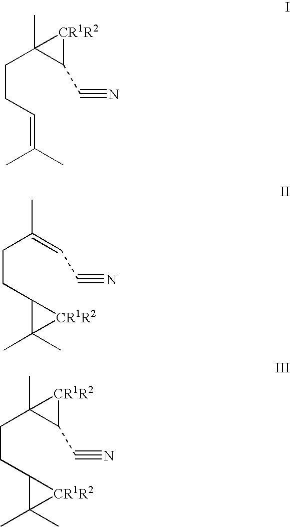

wherein R¹ and R² are each independently H or CH₃. R¹ and R² may be the same or different. In other words, both R¹ and R² may be H or one of R¹ and R² may be H and the other one may be methyl or R¹ and R² may both be methyl. These compounds are referred to hereinafter as “the compounds of the invention”.

For example, the compounds of the invention of formula I have the following stereoisomers.

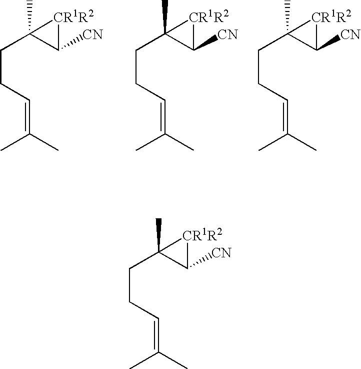

The compounds of the invention of formula II have the following stereoisomers.

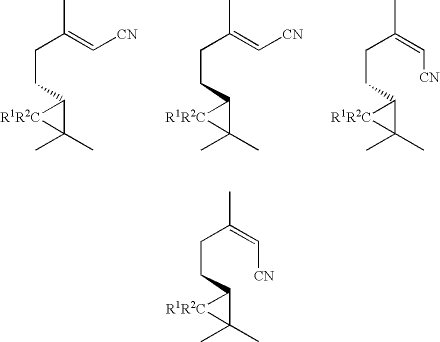

The compounds of the invention of formula III have the following stereoisomers.

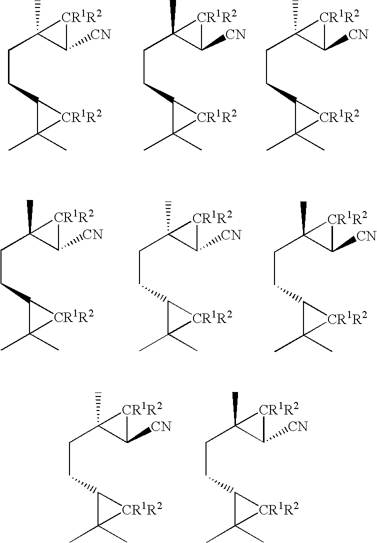

The compounds of the invention typically have improved physical and/or chemical properties relative to the conventional compounds on which they are based. For example, the compounds of the invention may have increased stability to high or low pH (i.e., acidic and/or alkaline media), and/or improved half-life, and/or lower likelihood of being potentially harmful to mammals such as humans (e.g. lower risk of causing allergic reactions), and/or reduced toxicity/genotoxicity such as carcinogenicity and/or increased odour intensity.

Examples of compounds of the invention are those in which R¹ and R² are H. Thus, specific examples of the compounds of formula I include the stereoisomers of 2-(4-methylpent-3-en-1-yl)cyclopropanecarbonitrile:

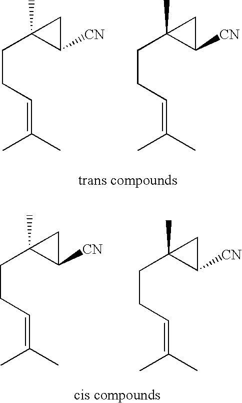

These compounds have interesting odour profiles. For example, the trans compounds in particular have a lemony odour and are acid stable.

Specific examples of compounds of formula II include the following stereoisomers.

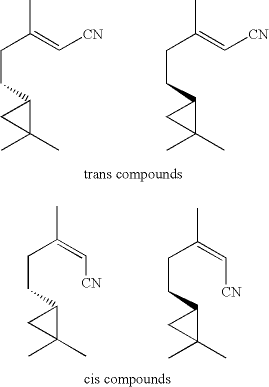

The present invention provides a method for preparing the a compound or mixture of compounds of formulae I, II or II comprising converting a corresponding aldehyde compound or mixture of compounds of formulae I″, II″ or III″

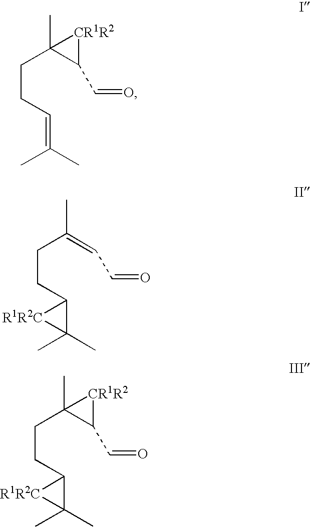

to the nitrile compounds of the invention (formulae I, II or III). This method will be described in more detail below.

The compounds of the invention of formula I or II can be prepared starting from the parent compounds, geraniol ((2E)-3,7-dimethyl-2,6-Octadien-1-ol) and/or nerol ((2Z)-3,7-dimethyl-,2,6-Octadien-1-ol):

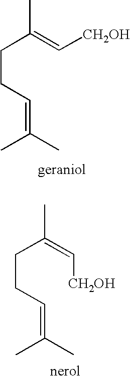

The first step of producing the compounds of formula I or II starting from geraniol and/or nerol is typically the monocyclopropanation of geraniol and/or nerol, illustrated as follows:

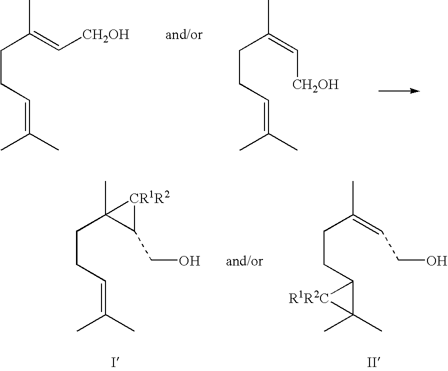

Any suitable method of cyclopropanation may be used. Suitable methods include carbenoid reactions such the Simmons-Smith cyclopropane synthesis (see for example Vogel's textbook of Practical Organic Chemistry 5^{th} Edition (1989) pp 1106-1108 or Solomon's Organic Chemistry 4^{th} Edition pp 346 and 347, published by John Wiley and Sons). The monocyclopropanation reaction using the Simmons-Smith synthesis can be directed to either the 2,3-cyclopropanated product (formula I′) or the 6,7-cyclopropanated product (formula II″) or both by selection of suitable reagent and/or conditions (see, for example, Stephenson, PhD thesis, University of Pittsburgh, 2004). Generally, however, the Simmons-Smith cyclopropanation of geraniol/nerol favours production of the 2,3-cyclopropanated product whereas Simmons-Smith cyclopropanation of geranial/neral favours production of the 6,7-cyclopropanated product.

Alternatively, the monocyclopropanated products shown above can be synthesized by subjecting geraniol and/or nerol to the haloform reaction to produce the dichloro or dibromo cyclopropyl derivative followed by dehalogenation with, e.g., lithium to provide the desired product. The haloform reaction may be used to prepare either 2,3- or 6,7-cyclopropanated products. Preferably, the haloform reaction is used to prepare 6,7-cyclopropanated products.

The Friedrichs reaction may also be used to prepare the 2,3-cyclopropanated product (formula I′) (see, for example, Friedrich & Lewis, J. Org. Chem., 1990, 55, 2491-2494). In this reaction, acetyl chloride is used to accelerate the cyclopropanation of an alkene with a 1,1,-dibromo or 1,1-diiodo alkyl such as dibromomethane or diiodomethane using zinc dust and copper (I) in ether. The Friedrichs reaction is preferred for preparing the compounds of formula I′ from geraniol/nerol.

Geraniol and nerol or geranial and neral may be cyclopropanated separately in order to produce the trans- or cis-compounds separately as required. Alternatively, a mixture of geraniol and nerol or geranial and neral may be cyclopropanated.

It is believed that the relative arrangement of the groups in geraniol/geranial and nerol/neral is maintained during the cyclopropanation reaction. Thus the use of geraniol alone will typically produce the trans-compounds (as in geraniol) only and the use of nerol alone will typically produce the cis-compounds (as in nerol) only. If a mixture of geraniol and nerol is used, the cyclopropanated produce will contain both the trans- and cis-compounds approximately in the proportions in which the starting material contained geraniol and nerol. This is illustrated below with reference to the 2,3-cyclopropanation (for example, using the Friedrichs reaction) to produce the compounds of formula I′.

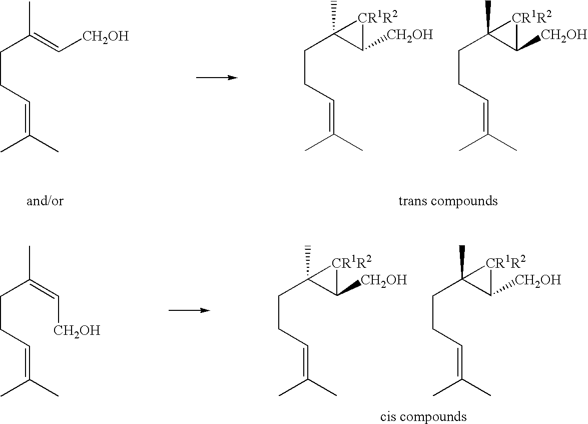

The remaining steps of the preparation of the monocyclopropanated compounds of the invention will be described with reference to the compounds of formula I. However, it will be appreciated that these steps may equally be applied to the compounds of formula II or III.

The second step of producing the compounds of formula I starting from geraniol and/or nerol is typically the oxidation of the monocyclopropanated alcohols of formula I′ to produce the corresponding aldehydes of formula I″, as shown below.

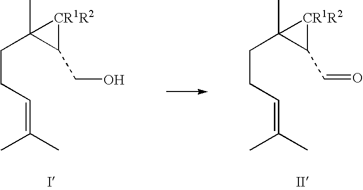

The alcohols can be converted to the aldehydes using any suitable method for the oxidation of an alcohol to form an aldehyde (for example as described in March “Advanced Organic Chemistry: Reactions, Mechanisms, and Structure”, 4^{th} Edition, John Wiley & Sons 1985, pages 1167 to 1171). One preferred method for producing aldehydes is the oxidation of the alcohols in a solution of dichloromethane using pyridinium dichromate.

The compounds of formula I″ or the corresponding 6,7-cyclopropanated compounds (formula II″) may also be produced in one step directly via the monocyclopropanation of geranial and/or neral (the mixture of geranial and neral being known as citral), as illustrated below. 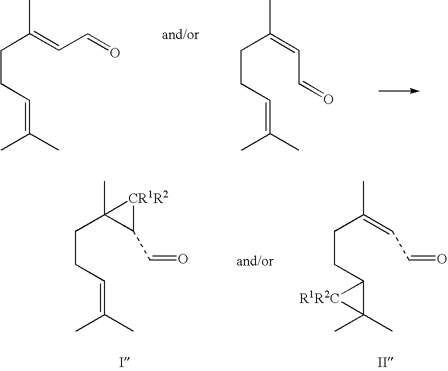
Any suitable cyclopropanation method may be used to roduce the compounds of formula I″ and/or II″ from geranial, neral or citral, as described above in relation to the cyclopropanation of geraniol and/or nerol.

For example, the Simmons-Smith cyclopropanation reaction is particularly suitable for use to produce compounds of formula II. One method which is suitable for preparing the compounds of formula I″ is by reaction of geranial and/or neral (or citral) with a suitable sulfoxonium ylide reagent. The use of sulfoxoium ylides in cyclopropanating reactions is described in for example March, “Advanced Organic Chemistry: Reactions, Mechanisms and Structure”, fourth edition (1992), John Wiley & Sons, Inc, page 872.

Suitable sulfoxonium ylides include

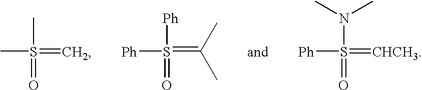

Thus, the compounds of formula I″ may be prepared by reaction of geranial and/or neral (or citral) as illustrated below.

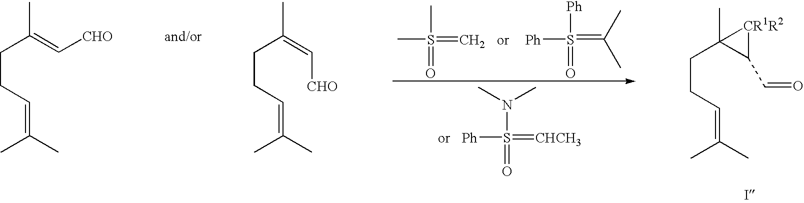

Dimethyloxosulfonium methylide can be obtained by deprotonation of trimethylsulfoxonium iodide with a base such as sodium hydride in any suitable solvent such as DMSO or DMF, preferably under an inert atmosphere (e.g. nitrogen or argon). Corresponding methods can be used to produce the other ylides.

The use of sulfoxoium ylides in cyclopropanating reactions is particularly suitable for the cyclopropanation of conjugated double bonds.

The trans-compounds of formula I may be prepared starting from geraniol (using the Friedrichs reaction for the cyclopropanation step) or starting from geranial (using the reaction with a sulfoxonium ylide) and the compounds of formula II are preferably formed starting from geranial (using the Simmons-Smith reaction for cyclopropanation).

Similarly, the cis-compounds of formula I may be prepared starting from nerol (using the Friedrichs reaction for the cyclopropanation) or starting from neral (using the reaction with a sulfoxonium ylide) and the compounds of formula II are preferably formed starting from neral (using the Simmons-Smith reaction for cyclopropanation). Specific examples of the monocyclopropanated aldehydes of formula I″ described above include 2-methyl-2-(4-methylpent-3-en-1-yl)cyclopropanecarbaldehyde:

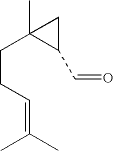

including all of its stereoisomers:

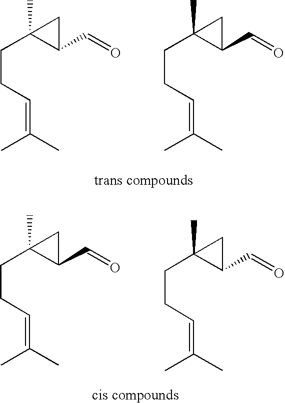

The third step of producing the compounds of formula I starting from geraniol and/or nerol (or the second step starting from geraniol and/or nerol) typically involves the conversion of the monocyclopropanated aldehydes of formula I″ to the analogous nitrile compounds of the invention of formula I, illustrated as follows:

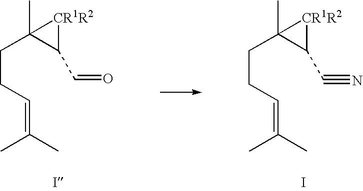

This may be achieved using any suitable method. One preferred method is via the corresponding aldoxime (—C═N—OH) (for example as described in March “Advanced Organic Chemistry: Reactions, Mechanisms, and Structure”, 4^{th} Edition, John Wiley & Sons 1992, pages 906-7 a). The aldoxime may be isolated and then dehydrated to form the nitrile or may be dehydrated in situ to form the nitrile. Suitable reagents for use in the dehydration of aldoximes are well known in the art and are described in for example March “Advanced Organic Chemistry: Reactions, Mechanisms, and Structure”, 4^{th} Edition, John Wiley & Sons 1992, pages 1038-9.

In summary, the present invention provides a method for the production of the compounds of the invention of formula I or II, which comprises converting monocyclopropanated aldehydes to the corresponding nitrites (of formula I or II). The monocyclopropanated aldehydes can be obtained by cyclopropanating geraniol and/or nerol, to produce the corresponding monocyclopropanated alcohols and oxidising the monocyclopropanated alcohols to produce the corresponding monocyclopropanated aldehydes. Alternatively, the monocyclopropanated aldehydes may be obtained by cyclopropanating geranial and/or neral. The reaction scheme starting from geraniol and/or nerol is summarised below.

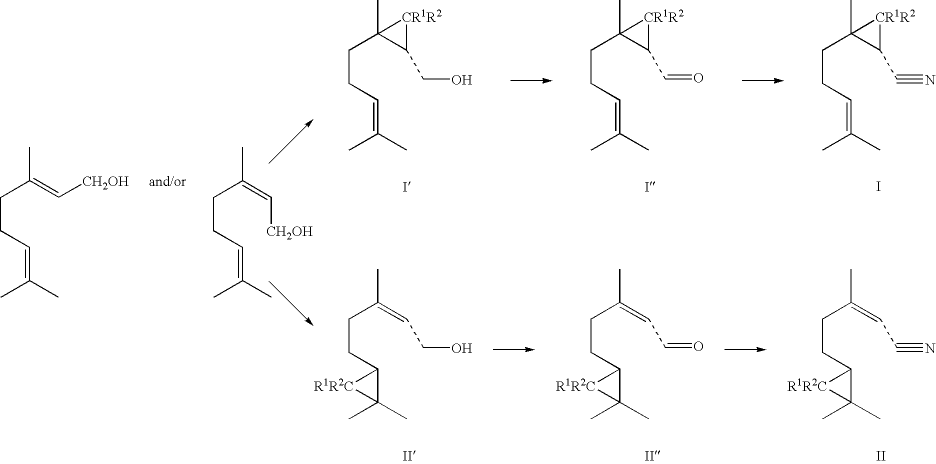

The reaction scheme starting from geranial and/or neral is summarised below.

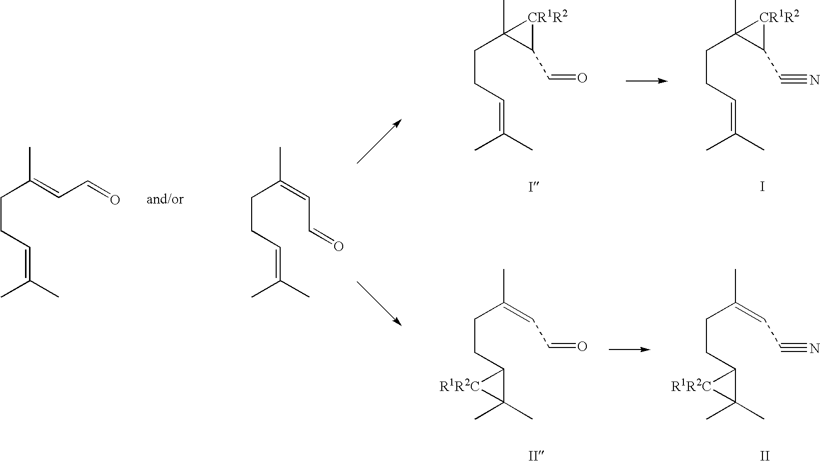

The methods of preparing the compounds of the invention proceed with retention of stereochemistry. For example, the trans compounds of formula I can be prepared starting from geraniol, as follows:

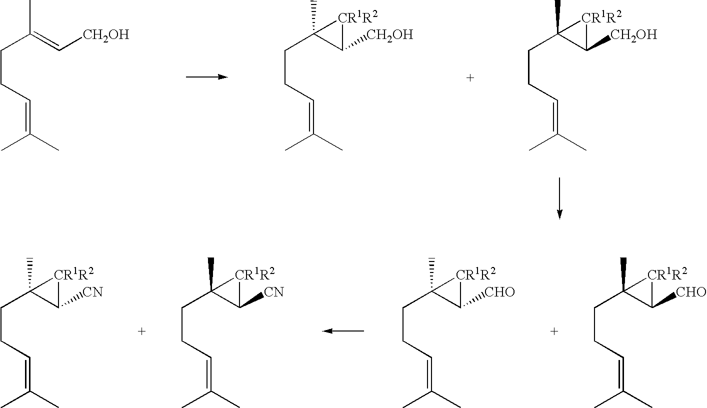

Alternatively, the cis compounds of formula I can be prepared starting from nerol, as illustrated below:

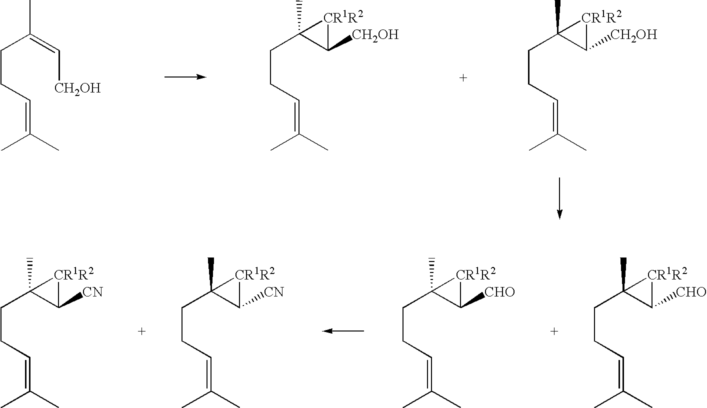

Similarly, the trans compounds of formula I can be prepared starting from geranial, as follows:

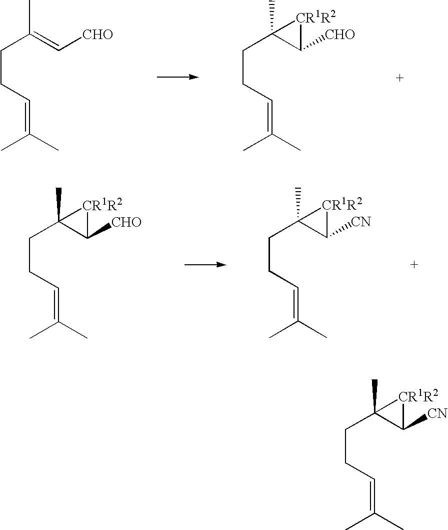

Alternatively, the cis compounds of formula I can be prepared starting from neral, as illustrated below:

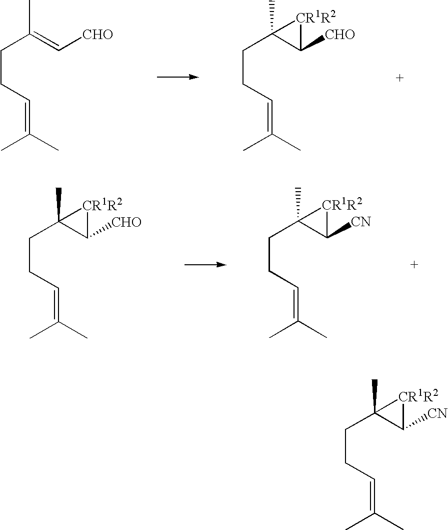

Alternatively, the trans or cis monocyclopropanated compounds of formula I may be prepared starting from a mixture of geraniol and nerol or geranial and neral. When a product containing a mixture of the trans- and cis-compounds is produced, the product may be used as a flavour or fragrance or for further reaction as a mixture or may be separated into the trans- and cis-nitrile compounds by any suitable method. Alternatively, the trans and cis products may be separated from each other at an appropriate point in the reaction scheme. For example, a mixture of the monocyclopropanated alcohols or aldehydes may be separated into their respective trans and cis products by any suitable method.

A preferred dicyclopropanated compound of the invention of formula III is 2-[2-(2,2-dimethylcyclopropyl)ethyl]cyclopropanecarbonitrile:

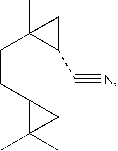

including all its stereoisomers:

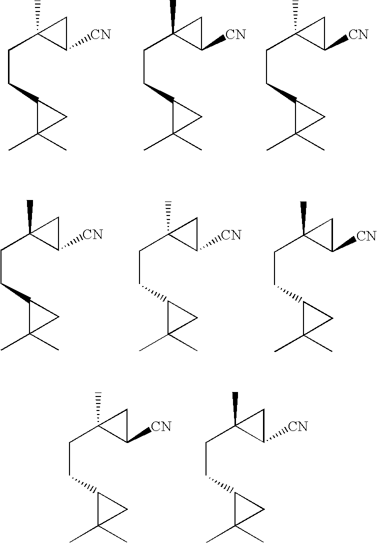

The compounds of formula III are typically prepared by converting the dicyclopropanated aldehydes to the corresponding nitriles (of formula III). The dicyclopropanated aldehydes can be prepared by dicyclopropanation of geraniol and/or nerol to produce compounds of formula (III′) followed by oxidising the dicyclopropanated alcohols to produce the corresponding dicyclopropanated aldehydes (formula III″). Alternatively, the dicyclopropanated aldehydes can be obtained by the dicyclopropanation of geranial and/or neral. The reaction scheme starting from geraniol and/or nerol is summarised below.

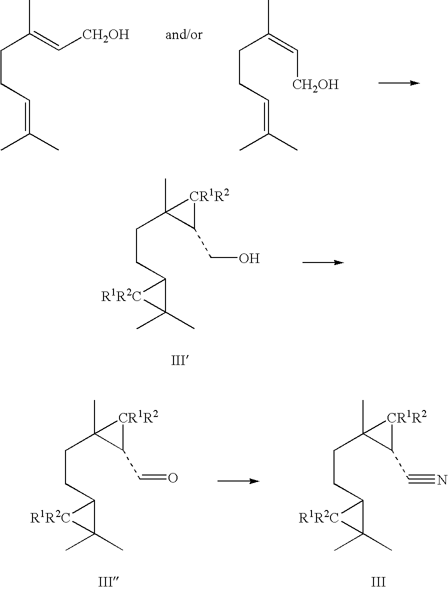

The reaction scheme starting from geranial and/or neral is summarised below.

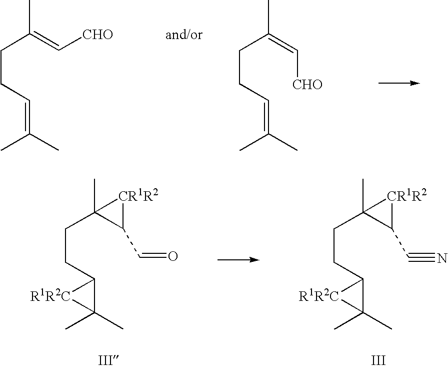

Any suitable method of cyclopropanation may be used for the dicyclopropanation of geraniol/nerol and geranial/neral. The methods described above for the monocyclopropanation reactions are also suitable for the dicyclopropanation. It will be appreciated that some alteration of the reaction time and conditions of the cyclopropanation reactions would be required to encourage the production of the dicyclopropanated product. The person of ordinary skill in the art would be readily able to select suitable reaction conditions. Typically, a higher temperature and/or a longer reaction time and an alteration of the stoichiometry of the reagents is required in order to favour production of the dicyclopropanated product.

Alternatively, the production of a dicyclopropanated compound may take place in several steps. For example, starting with geraniol and/or nerol, a compound of formula I′ may be produced using the Friedrichs reaction. The alcohol group may then be converted to an aldehyde group to give the compound of formula I″, which may then be converted to the dicyclopropanated aldehyde product (formula III″) using the Simmons-Smith reaction.

It will be appreciated that the cyclopropanation reactions described above may often produce a mixture of mono and dicyclopropanated products. These products typically have slightly different molecular weights. As a result, if necessary, they can be separated using simple distillation techniques. Any such distillation would preferably be conducted on the cyclopropanated alcohols or nitrites. The boiling point of the monocyclopropanated alcohol is calculated to be about 229±9° C. and that of the dicyclopropanated alcohol to be about 208±8° C. The boiling point of the monocyclopropanated nitrile is calculated to be about 244±9° C. and that of the dicyclopropanated nitrile to be about 254±9° C.

As stereochemistry is retained in the methods for producing the compounds of formula III described above, different stereoisomers can be obtained depending on which starting material is dicyclopropanated. Thus, the methods described above starting from geraniol or geranial ultimately results in the four corresponding trans compounds:

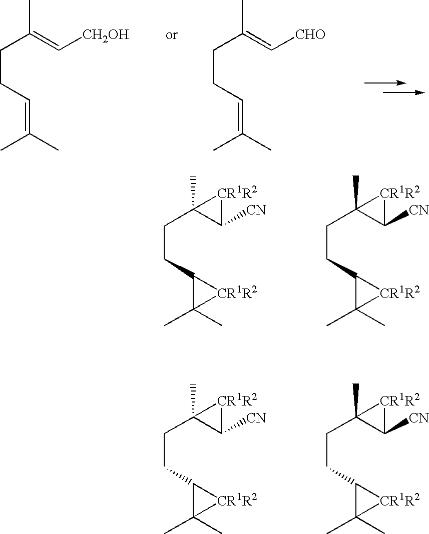

The same methods starting from nerol or neral ultimately produce the corresponding cis compounds:

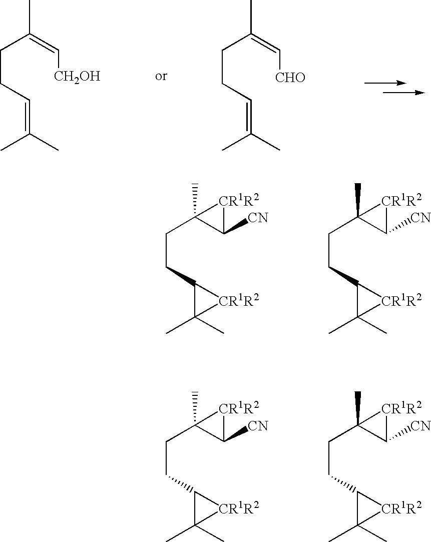

The compounds of formula III may also be prepared by cyclopropanation of the compounds formula I or II, as summarised below.

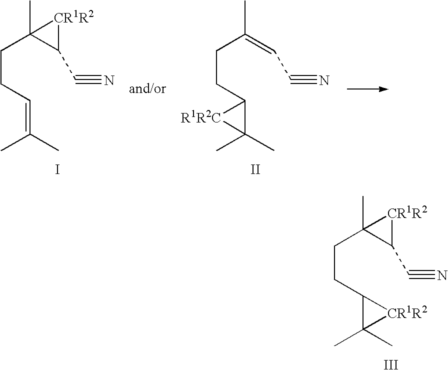

Any suitable method of cyclopropanation may be used for converting the monocyclopropanated compounds of formula I or II to the dicyclopropanated compounds of formula III. Generally, however, cyclopropanation of compounds of formula I is more facile than cyclopropanation of compounds of formula II. Typically, the compounds of formula I may be converted to the compounds of formula III using the Simmons-Smith or haloform reaction described hereinbefore.

The compounds of the invention of formulae I, II and III may also be prepared by direct nitrilation of the corresponding alcohols of formulae I′, II′ and III′, respectively. This may be achieved by any suitable means, for example by treatment of the compounds of formulae I′, II′ or III′ with NaCN, Me₃SiCl and a catalytic amount of NaI in DMF-MeCN (see, for example, March, “Advanced Organic Chemistry: Reactions, Mechanisms and Structure”, fourth edition (1992), John Wiley & Sons, Inc, page 872).

The compounds of the invention may also be prepared by cyclopropanation of 3,7-dimethyl-2,6-octadienenitrile, which is commercially available (e.g., Aldrich) whose structure is shown below.

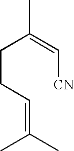

For example, 3,7-dimethyl-2,6-octadienenitrile may be monocyclopropanated at the 2,3- or 6,7-double bond to generate the compounds of the invention of formula I or II, respectively. Alternatively, the 3,7-dimethyl-2,6-octadienenitrile may be dicyclopropanated at both the 2,3- and 6,7-double bonds to generate the compounds of the invention of formula III.

Compounds of the invention may exhibit tautomerism. All tautomeric forms and mixtures thereof are included within the scope of the invention.

The compounds of the invention contain one or more asymmetric carbon atoms and therefore exhibit optical and/or diastereoisomerism. The compounds of the invention may also contain double bonds existing as E (entgegen) and Z (zusammen) geometric isomers about each individual double bond. As used herein, a dashed bond indicates that the relevant carbon atom is in the R and S configuration or that the relevant double bond is in the E or Z configuration. Additionally, dashed or bold wedge bonds indicate that the relevant carbon atom is in the R or S configuration.

The compounds of the invention exhibit optical and/or diastereoisomerism. Diastereoisomers may be separated using conventional techniques, e.g. chromatography or fractional crystallisation. The compounds of the invention may be used as a racemic mixture of stereoisomers or may be separated into individual isomers which may then be used separately in pre-selected ratios. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst, all under conditions known to the skilled person.

The present invention provides for the use of the compounds of the invention and mixtures thereof as a flavor and/or fragrance. For example, the compounds of the invention typically have a citrus (e.g. lemony) odour and are acid/base stable.

The present invention also provides compositions, products, preparations or articles containing a compound or mixture of compounds of the invention as described above.

The present invention also provides methods to confer, improve, enhance or modify the taste or flavor property of a composition, product, preparation or article which comprises adding thereto a flavor effective amount of a composition or mixture of compounds of the invention as described above.

A method to confer, improve, enhance or modify the aroma, fragrance or odor characteristics of compositions, products, preparations or articles which comprises adding thereto an aroma, fragrance or odor effective amount of a composition or mixture of compounds of the invention as described above is also provided.

The compounds of the invention can be included in virtually any article of manufacture that can include fragrance or flavorant compounds. Examples include hypochlorite (bleach) compositions, detergents, flavorings and fragrances, beverages, including alcoholic beverages, and the like. The compounds of the invention can be used in applications like soaps, shampoos, denture cleanser tablets, body deodorants and antiperspirants, solid or liquid detergents for treating textiles, fabric softeners, detergent compositions and/or all-purpose cleaners for cleaning dishes or various surfaces, for both household and industrial use and candles. Of course, the use of the compounds is not limited to the above-mentioned products, as they may be used in other current uses in perfumery, namely the perfuming of soaps and shower gels, hygiene or hair-care products, as well as of body deodorants, air fresheners and cosmetic preparations, and even in fine perfumery, namely in perfumes and colognes. The compositions of the invention are particularly suited to bleach compositions.

The compounds of the invention also find utility in foods, flavorings, beverages such as beer and soda, denture cleansers (tablets), flavored orally-delivered products such as lozenges, candies, chewing gums, matrices, pharmaceuticals and the like. These uses are described in more detail below.

The compounds of the invention can be used as perfuming ingredients, as single compounds or as mixtures thereof. The compounds can be used in their pure state or as mixtures, without added components. The olfactive characteristics of the individual compounds are also present in mixtures thereof, and mixtures of these compounds can be used as perfuming ingredients. This may be particularly advantageous where separation and/or purification steps can be avoided by using compound mixtures.

In all of the above applications, the compounds of the invention can be used alone, in admixture with each other, or in admixture with other perfuming ingredients, solvents or adjuvants of current use in the art. The nature and the variety of these co-ingredients do not require a more detailed description here, which, moreover, would not be exhaustive, and the person skilled in the art will be able to choose the latter through their general knowledge and as a function of the nature of the product to be perfumed and of the desired olfactive effect.

These perfuming ingredients typically belong to chemical classes as varied as alcohols, aldehydes, ketones, esters, ethers, acetates, nitrites, terpene hydrocarbons, sulfur- and nitrogen containing heterocyclic compounds, as well as essential oils of natural or synthetic origin. A large number of these ingredients described in reference textbooks such as the book of S. Arctander, Perfume and Flavor Chemicals, 1969, Montclair, N.J., USA, the contents of which are hereby incorporated by reference in its entirety, or its more recent versions, or in other works of similar nature.

The proportions in which the compounds of the invention can be incorporated in the various products vary within a large range of values. These values depend on the nature of the article or product that one desires to perfume and the odor effect searched for, as well as on the nature of the co-ingredients in a given composition when the compounds are used in admixture with perfuming co-ingredients, solvents or adjuvants of current use in the art.

As an example, the compounds of the invention are typically present at concentrations between about 0.01 and about 30%, or even more, by weight of these compounds relative to the weight of the composition, product or article in which they are incorporated. It will be appreciated that the amount by weight of a compound of the invention in a particular composition or product will depend on the nature of the composition. For example, a washing powder will typically contain less than 1% by weight of a compound of the invention while a fine fragrance may contain more than 20% by weight of a compound of the invention.

The compounds may be used in detergents such as those containing bleaching agents and activators such as, for example, tetraacetylethylenediamine (TAED), hypohalites, in particular hypochlorite, peroxygenated bleaching agents such as, for example, perborates, etc. The compounds can also be used in body deodorants and antiperspirants, for example, those containing aluminum salts. These aspects are described in more detail below.

In addition to the compounds of the invention, the compositions described herein may include a detersive surfactant and optionally, one or more additional detergent ingredients, including materials for assisting or enhancing cleaning performance, treatment of the substrate to be cleaned, or to modify the aesthetics of the detergent composition (e.g. perfumes, colorants, dyes, etc.). Non-limiting examples of synthetic detersive surfactants useful herein typically at levels from about 0.5% to about 90%, by weight, include the conventional C₁₋₁₈ alkyl benzene sulfonates (“LAS”) and primary, branch-chain and random C₁₀₋₂₀ alkyl sulfates (“AS”), and the like. Preferred compositions incorporating only synthetic detergents have a detergent level of from about 0.5% to 50%. Compositions containing soap preferably comprise from about 10% to about 90% soap.

The compositions described herein can contain other ingredients such as enzymes, bleaches, fabric softening agents, dye transfer inhibitors, suds suppressors, and chelating agents, all well known within the art.

The compounds of the invention can be incorporated into beverages and impart various flavorings to the beverages. The beverage composition can be a cola beverage composition, and can also be coffee, tea, dairy beverage, fruit juice drink, orange drink, lemon-lime drink, beer, malt beverages, or other flavored beverage. The beverages can be in liquid or powdered form. The beverage compositions can also include one or more flavoring agents; artificial colorants; vitamin additives; preservatives; caffeine additives; water; acidulants; thickeners; buffering agents; emulsifiers; and/or fruit juice concentrates.

Artificial colorants that may be used include caramel color, yellow 6 and yellow 5. Useful vitamin additives include vitamin B2, vitamin B6, vitamin B12, vitamin C (ascorbic acid), niacin, pantothenic acid, biotin and folic acid. Suitable preservatives include sodium or potassium benzoate. Salts that may be used include sodium, potassium and magnesium chloride. Exemplary emulsifiers are gum arabic and purity gum, and a useful thickener is pectin. Suitable acidulants include citric, phosphoric and malic acid, and potential buffering agents include sodium and potassium citrate.

The beverage may, for example, be a carbonated cola beverage. The pH is generally about 2.8 and the following ingredients can be used to make the syrup for these compositions: Flavor Concentrate, including one or more of the compounds of the invention herein (22.22 ml), 80% Phosphoric Acid (5.55 g), Citric Acid (0.267 g), Caffeine (1.24 g), artificial sweetener, sugar or corn syrup (to taste, depending on the actual sweetener) and Potassium Citrate (4.07 g). The beverage composition can be prepared, for example, by mixing the foregoing syrup with carbonated water in a proportion of 50 ml syrup to 250 ml of carbonated water.

Flavored food and pharmaceutical compositions including one or more of the compounds of the invention can also be prepared. The compounds of the invention can be incorporated into conventional foodstuffs using techniques well known to those of skill in the art. Alternatively, the compounds can be incorporated within polymeric particles, which can, in turn, be dispersed within and/or over a surface of an orally-deliverable matrix material, which is usually a solid or semi-solid substrate. When used in chewable compositions, the compounds of the invention can be released into the orally-deliverable polymeric matrix material as the composition is chewed and held in the mouth, thus prolonging the flavor of the composition. In the case of dried powders and mixes, the flavor can be made available as the product is consumed or be released into the matrix material as the composition is further processed. When two flavors are combined with the polymeric particles, the relative amounts of the additives can be selected to provide simultaneous release and exhaustion of the compounds.

Flavored compositions of the invention may include an orally-deliverable matrix material; a plurality of water insoluble polymeric particles dispersed in the orally-deliverable matrix material, where the polymeric particles individually define networks of internal pores and are non-degradable in the digestive tract; and one or more compounds of the invention entrapped within the internal pore networks. The compounds of the invention are released as the matrix is chewed, dissolved in the mouth, or undergoes further processing selected from the group consisting of liquid addition, dry blending, stirring, mixing, heating, baking, and cooking. The orally-deliverable matrix material can be selected from the group consisting of gums, latex materials, crystallized sugars, amorphous sugars, fondants, nougats, jams, jellies, pastes, powders, dry blends, dehydrated food mixes, baked goods, batters, doughs, tablets, and lozenges.

A flavorless gum base can be combined with a compound or a mixture of compounds of the invention to a desired flavor concentration. In one method for producing such gum based products a blade mixer is heated to about 110° F., the gum base is preheated so that it is softened, and the gum base is then added to the mixer and allowed to mix for approximately 30 seconds. The compound or compounds of the invention are then added to the mixer and mixed for a suitable amount of time. The gum can be then removed from the mixer and rolled to stick thickness on waxed paper while warm.

The compounds of the invention may be incorporated into a system that can release a fragrance in a controlled manner. These include substrates such as air fresheners, laundry detergents, fabric softeners, deodorants, lotions, and other household items. The fragrances are generally one or more derivatives of essential oils as described herein, each present in different quantities. U.S. Pat. No. 4,587,129, the contents of which are hereby incorporated by reference in their entirety, describes a method for preparing gel articles that contain up to 90% by weight of fragrance or perfume oils. The gels are prepared from a polymer having a hydroxy (lower alkoxy) 2-alkeneoate, a hydroxy (lower alkoxy) lower alkyl 2-alkeneoate, or a hydroxy poly (lower alkoxy) lower alkyl 2-alkeneoate and a polyethylenically unsaturated crosslinking agent. These materials have continuous slow release properties, i.e. they release the fragrance component continuously over a long period of time. Advantageously, all or a portion of those derivatives that include an aldehyde group can be modified to include an acetal group, which can cause the formulations to release fragrance over a period of time as the acetal hydrolyzes to form the aldehyde compound.

The present invention is illustrated by the following non-limiting examples.

### Synthesis of (2-methyl-2-(4-methylpent-3-enyl)cyclopropyl)methanol

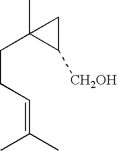

Dibromomethane (13.5 ml, 0.19 mol) and acetyl chloride (1.2 ml, 19.2 mmol) were added to a suspension of zinc dust (38 g, 0.58 mol) and copper chloride (5.74 g, 0.06 mol) in diethyl ether (200 ml) at room temperature. The mixture was stirred 10 minutes before Geraniol/Nerol (30 g, 0.19 mol), and dibromethane (2.25 ml, 32.4 mmol) were added dropwise. The reaction was kept below 20° C. during and after the addition. The reaction mixture was stirred overnight, and then poured into an ammonium chloride solution at 0° C. The aqueous phase was extracted with ethyl acetate (3×500 ml). The organic phases were dried over magnesium sulphate and evaporated. Purification by silica gel chromatography with a gradient of 2-5% ethyl acetate/hexane provided the desired compound as colourless oil (25.3 g, 0.15 mol) in 80% yield.

Note: The temperature of the reaction mixture during the addition of geraniol should be kept under 20° C. to avoid any formation of di-cyclopropane adduct. Three equivalents of Zinc should be used to obtain a full conversion, as it is very difficult to separate the starting material and the desired product by column chromatography or distillation.

### Synthesis of 2-methyl-2-(4-methylpent-3-enyl)cyclopropanecarbaldehyde

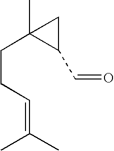

A solution of 2-methyl-2-(4-methylpent-3-enyl)cyclopropyl)methanol (33 g, 0.19 mol) in dichloromethane (100 ml) was added to a solution of pyridinium dichromate (110.8 g, 0.294 mol) in dichloromethane (1 L) at 0° C. The reaction mixture was stirred at 0° C. for 30 minutes then at room temperature overnight. The mixture was diluted in diethyl ether (500 ml) and filtered through celite. After evaporation of the solvent the crude product was purified by silica gel chromatography eluting with dichloromethane provided the desired compound as colourless oil (16.7 g, 0.1 mol) in 53% yield.

### Synthesis of 2-(4-methylpent-3-en-1-yl)cyclopropanecarbonitrile

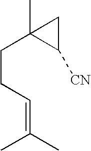

2-Methyl-2-(4-methylpent-3-enyl)cyclopropanecarbaldehyde (1 g, 6 mmol), hydroxylamine hydrochloride (586 mg, 8.4 mmol) and pyridine (0.48 ml, 6.02 mmol) in ethanol (10 ml) were stirred at room temperature for 4 hours. After evaporation of the solvent the crude product was taken in ethyl acetate. The organic phase was washed with water, 1N HCl, brine, dried over magnesium sulfate and evaporated to give the corresponding oxime (0.7 g).

A mixture of the oxime (0.3 g, 1.6 mmol), sodium acetate (0.54 g, 6.6 mmol) and acetic anhydride (0.3 ml, 3.3 mmol) was stirred and heated at 40° C. for 6 hours. The reaction mixture was poured into water and extracted with ethyl acetate (3×50 ml). The organic phases were dried over magnesium sulphate and evaporated. Purification by silica gel chromatography eluting with a gradient of 1-2% ethyl acetate/hexane provided the desired compound as colourless oil (110 mg, 0.6 mmol) in 42% yield.

Having hereby disclosed the subject matter of the present invention, it should be apparent that many modifications, substitutions, and variations of the present invention are possible in light thereof. It is to be understood that the present invention can be practiced other than as specifically described. Such modifications, substitutions and variations are intended to be within the scope of the present application.

1. One or more compounds of formulae: 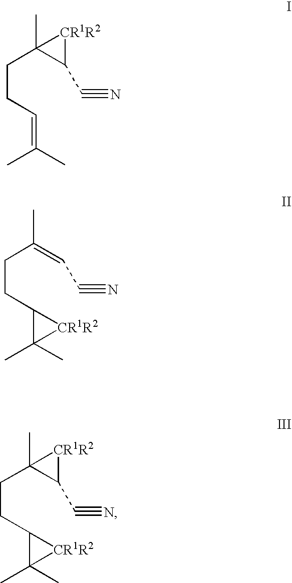
wherein R¹ and R² are each independently H or CH₃.

2. One or more compounds according to claim 1, formula I, of the following formulae:

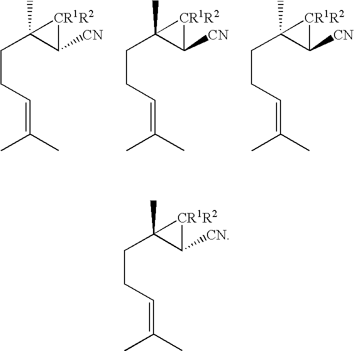

3. One or more compounds according to claim 2 of the following formulae:

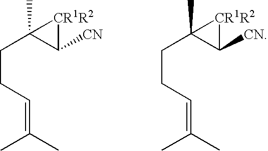

4. One or more compounds according to claim 1, formula II, having the following formulae:

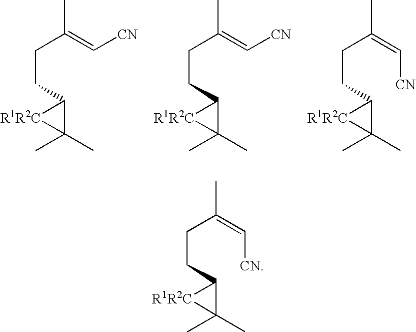

5. One or more compounds according to claim 1, formula III, having the following formulae:

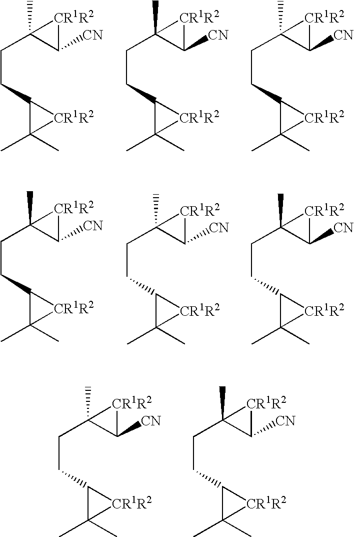

6. One or more compounds according to claim 5 having one or more of the following formulae:

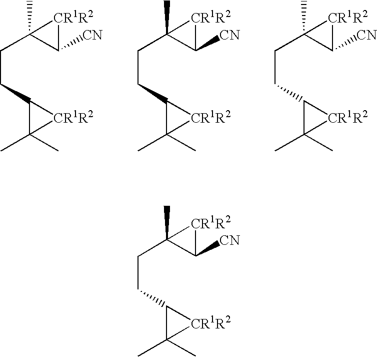

7. At least one compound according to claim 1 wherein R¹ and R² represent H.

8. A method for preparing a compound or mixture of compounds comprising:
converting at least one aldehyde compound of formulae 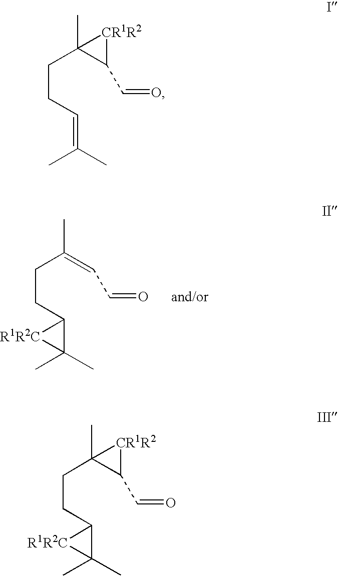
to a compound or mixture of compounds of formulae: 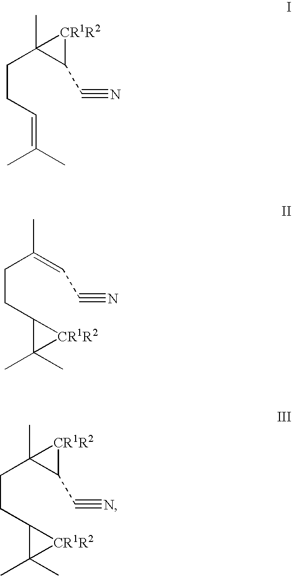
wherein R¹ and R² are each independently H or CH₃.

9. A method according to claim 8 comprising cyclopropanating geranial and/or neral to produce the compound or mixture of compounds of formulae I″, II″ and/or III″.

10. A method according to claim 8 comprising oxidizing a compound or mixture of compounds of formulae 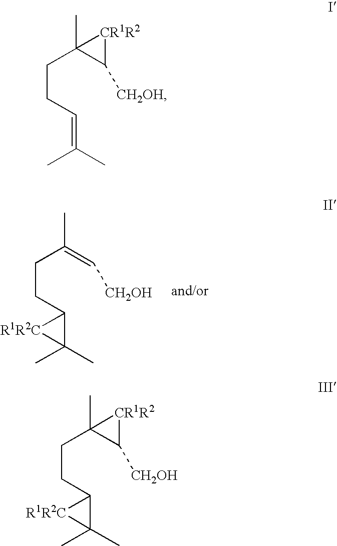
to produce at least one compound of formulae I″, II″ or III″.

11. A method according to claim 10 comprising cyclopropanating geraniol and/or nerol to produce the compound or mixture of compounds of formulae I′ or II′ or III′.

12. A method according to claim 8 comprising cyclopropanating the compound or mixture of compounds of formulae I or II to produce the compound of formula III.

13. A method for preparing one or more compounds according to claim 1 comprising cyclopropanating 3,7-dimethyl-2,6-octadienenitrile.

14. A flavour and/or fragrance composition comprising a compound or a mixture of compounds as defined in claim 1.

15. A substrate treated with a compound or a mixture of compounds as defined in claim 1.

16. A method for treating a substrate to impart flavourant/fragrance releasing characteristics thereto comprising applying to the substrate a compound or a mixture of compounds as defined in claim 1.

17. A composition having aroma, fragrance or odour releasing characteristics containing a compound or mixture of compounds as defined in claims 1, in admixture with at least one of an additional perfuming ingredient, a solvent, and an adjuvant.

18. A composition according to claim 17 in the form of a perfume, fragrance or cologne, a soap, a bath or shower gel, a hair care product, a cosmetic preparation, a body odorant, deodorant or antiperspirant, an air freshener, a liquid or solid fabric detergent or softener, bleach product, disinfectant or an all-purpose household or industrial cleaner.

19. A composition according to claim 18, wherein the compound or mixture of compounds is in admixture with at least one bleach ingredient.

20. A composition according claim 18, wherein the compound or mixture of compounds is in admixture with at least one disinfectant ingredient.

21. A composition according to claim 18 in the form of a body odorant, deodorant or antiperspirant wherein the compound or mixture of compounds is in admixture with at least one body odorant, deodorant or antiperspirant ingredient.

22. A composition according to claim 1 in the form of a beverage, which contains at least one beverage ingredient.

23. A composition according to claim 1 in the form of a flavouring, which contains at least one flavouring ingredient.

24. A composition according to claim 1 in the form of a food, which contains at least one food ingredient.

25. A composition according to claim 1 in the form of a chewing gum, which contains at least one chewing gum ingredient.

26. A composition according to claim 1 in the form of a pharmaceutical, which contains at least one pharmaceutical ingredient.

27. A composition according to claim 1 in the form of an orally-deliverable matrix material which contains at least one matrix material ingredient.

28. A method to confer, improve, enhance or modify a taste or flavour property of a composition which comprises adding thereto a flavour effective amount of a compound or mixture of compounds as defined in claim 1.

29. A method according to claim 28, wherein said composition is in the form of a beverage, a flavouring, a food, a chewing gum, a pharmaceutical or an orally deliverable matrix.

30. A method to confer, improve, enhance or modify an aroma, fragrance or odour characteristics of a composition which comprises adding thereto an aroma, fragrance or odour effective amount of a compound or mixture of compounds as defined in claim 1.

31. A method according to claim 30 wherein said composition is in the form of a perfume, a body odorant, deodorant or antiperspirant, a detergent, a bleach product or a disinfectant.

32. An article of manufacture comprising packaging material and an aroma, odour, fragrance, taste or flavour enhancing agent contained within the packaging material, wherein the agent is effective for the enhancement of the aroma, odour, fragrance, taste or flavour of a composition to which it is added, and wherein the packaging material comprises a label which indicates that the agent can be used for enhancing aroma, odour, fragrance, taste or flavour, and wherein the agent is a compound or mixture of compounds as defined in claim 1.
